(19) United States
(12) Patent Application Publication
(10) Pub. No.: US 2012/0107300 A1
(43) Pub. Date: May 3, 2012

(54) Cannabinoid Compositions and Methods

(76) Inventor: Jeffrey Nathan Schirripa, Kinnelon, NJ (US)

(21) Appl. No.: 12/917,427

(22) Filed: Nov. 1, 2010

(51) Int. Cl. A61K 38/48; A61K 31/7068; A61K 36/38; A61K 36/906; A61K 36/258; A61K 36/77; A61K 31/522; A61K 31/66; A61K 31/185; A61K 31/352; A61K 31/343; A61K 31/35; A61K 36/02; A61K 36/185; A61K 31/14; A61K 31/7052; A61K 36/31; A61K 31/197; A61K 36/889; A61K 31/198; A61K 31/685; A61K 38/22; A61K 31/55; A61K 31/439; A61K 31/675; A61K 31/404; A61K 31/4985; A61K 31/34

(52) U.S. Cl. 424/94.65; 514/474; 514/49; 424/730; 424/756; 424/728; 424/725; 514/263.31; 514/77; 514/578; 514/454; 514/468; 514/456; 514/453; 424/195.17; 424/776; 424/769; 514/642; 514/52; 424/755; 514/561; 424/727; 514/567; 514/78; 514/9.7; 514/215; 514/295; 514/563; 514/89; 514/419; 514/249

(57) ABSTRACT

Cannabinoid compositions and methods of administering said cannabinoid compositions are disclosed. The cannabinoid compositions disclosed herein include compounds which have been strategically selected to eliminate, or least mitigate, one of the negative side effects associated with consuming cannabinoids. A daytime formula includes compounds aimed at nullifying the negative side effects that cannabinoid consumption has on a patient's daily operations and activities. A nighttime formula includes compounds aimed at nullifying the negative side effects that cannabinoid consumption has a patient's sleep.

### BACKGROUND

1. Technical Field

The present invention relates to cannabinoid compositions, as well as to methods for producing and administering the cannabinoid compositions.

2. Description of the prior art

Medical marijuana is used to treat a vast array of medical conditions. Unfortunately, a number of different adverse side effects are associated with administering marijuana, or other substances which include cannabinoids. For example, administering marijuana tends to make patients feel lethargic and anxious, and also tends to decrease motivation, productivity and concentration. Moreover, patients experience impaired memory and communication skills. Even further, marijuana consumption negatively affects a patient's sleep patterns by decreasing levels of slow wave sleep (SWS) and rapid eye movement (REM) sleep.

In addition to the side effects mentioned above, a patient who consumes marijuana may experience additional adverse side effects related to the particular manner in which marijuana is administered. For example, it is commonly known that smoking marijuana can be harmful to a patient's throat or lungs. In addition, smoking marijuana increases the production of phlegm, increases the risk of lung infection, decreases a patient's lung capacity and lowers oxygen levels in a patient's blood.

Given the seriousness of the side effects associated with smoking marijuana, it may be preferred to administer marijuana orally (e.g., through digestion or sublingual absorption). However, orally consuming marijuana also has some disadvantages. For example, patients who orally consume marijuana tend to experience extreme lethargy (much more than when marijuana is consumed by smoking or other methods). In addition, when marijuana is ingested, it takes a relatively long time before its beneficial effects are experienced by a patient.

### SUMMARY OF THE INVENTION

The present invention relates to cannabinoid compositions and methods of administering said cannabinoid compositions. The cannabinoid compositions disclosed herein include compounds which have been strategically selected to eliminate, or least mitigate, one or more of the negative side effects associated with consuming cannabinoids. A daytime formula includes compounds aimed at nullifying the negative side effects that cannabinoid consumption has on a patient's daily operations and activities. A nighttime formula includes compounds aimed at nullifying the negative side effects that cannabinoid consumption has on a patient's sleep.

In accordance with the present principles, a composition comprises at least one cannabinoid and at least one of the following compounds: vitamin B complex, vitamin C, citicoline, guarana, L-Alpha Glycerylphosphorylcholine, ginseng, gingko biloba, taurine, caffeine, eleuthero, ginger, St. Johns wart, spirulina, kola nut, yerba mate, schisandra, bromelain, choline, maca root, 1-ornithine, lecithin, rhodiala, ornithine alpha ketoglutarate, acai, tyrosine, centella asiatica or rosemary.

In accordance with the present principles, a second composition comprises at least one cannabinoid and at least one of the following compounds: ginger, melatonin, galanthamine, huperzine A, vitamin B5, vitamin B6, valerian root, 5-Hydroxytryptophan, folic acid, 1-arginine, choline, spirulina, damiana, phenylalanine, bromelain, lecithin, ornithine alpha ketoglutarate, acai, tyrosine and rosemary.

In accordance with the present principles, a method for administering a cannabinoid composition is also disclosed. The method includes the step of administering a therapeutically effective amount of a cannabinoid composition to a patient. The cannabinoid composition administered comprises at least one cannabinoid and at least one of the following compounds: vitamin B complex, vitamin C, citicoline, guarana, L-Alpha Glycerylphosphorylcholine, ginseng, gingko biloba, taurine, caffeine, eleuthero, ginger, St. Johns wart, spirulina, kola nut, yerba mate, schisandra, bromelain, choline, maca root, 1-ornithine, lecithin, rhodiala, ornithine alpha ketoglutarate, acai, tyrosine or rosemary.

Other objects and features of the present principles will become apparent from the following detailed description.

### DETAILED DESCRIPTION

In accordance with the present principles, compositions are disclosed for a product which combines various compounds with a cannabinoid. Each of the compounds included in these compositions has been selectively chosen either to eliminate, or at least mitigate, one or more of the negative side effects associated with the consumption of cannabinoids, such as tetrahydrocannabinol (THC).

The different herbs and vitamins included may vary with respect to different embodiments. In one embodiment, a “daytime formula” includes ingredients which serve to increase a patient's energy, concentration and motivation, while enhancing the patient's memory and mood. In another embodiment, a “nighttime formula” includes ingredients that serve to improve a patient's quality of sleep and to reduce anxiety experienced by the patient. In addition, all of the compositions disclosed herein may additionally include ingredients which assist with digestion and absorption of cannabinoids and other compounds.

It is recognized that cannabinoids are commonly administered to patients who have contracted cancer or who are undergoing cancer treatments. Therefore, the compositions herein may include ingredients which combat the negative side affects associated with treating cancer treatments. For examples, ingredients may be included which assist in absorption of the compositions, which serve to boost a patient's immune system or which protect a patient from the radiation and chemicals used in a variety of different cancer treatments.

The compositions described herein may be incorporated into a variety of different products. In certain embodiments, the present principles may be incorporated into medications in any number of forms (e.g., pill form, liquid form, etc.). In other embodiments, the compositions described herein may be incorporated into edible products (e.g., brownies, cake, etc.) or drinks (e.g., tea, juice, etc.). In addition, the compositions described herein may also be included in intermediary products (e.g., butter, oil, etc.) which are used to produce other edible products.

In a preferred embodiment, the compositions described herein are incorporated in a lollipop/sucker. Embodying the compositions in a lollipop/sucker permits for easy sublingual absorption. Sublingual absorption is the preferred method of consumption because it avoids the unnecessary health risks associated with smoking (e.g., risk of lung infection, risk of contracting cancer, etc.) and permits the beneficial effects of the cannabinoid compositions to be felt more quickly than with digestion of cannabinoid compositions.

Upon reading the description provided herein, those skilled in the art will be able to devise various arrangements that embody the present principles despite the fact that these arrangements are not explicitly described or shown herein. These various arrangements are to be considered within the scope of the present invention. Moreover, it should be noted that all examples included in this description are merely provided to aid the reader in understanding the present principles, and that the present principles are not limited to the explicit examples described herein. In addition, it should further be understood that all information provided herein which describes particular embodiments, or aspects of particular embodiments, is intended to encompass all currently known equivalents as well as any and all equivalents developed in the future.

As used herein, the term “cannabinoid” refers to a chemical or a group of chemicals which activate the body's cannabinoid receptors (e.g., CB₁ and CB₂ receptors). The term includes phytocannabinoids, endogenous cannabinoids and synthetic cannabinoids.

Phytocannabinoids (also known as natural cannabinoids, herbal cannabinoids, or classical cannabinoids) are compounds that are found in Cannabis plants (e.g., Cannabis sativa, Cannabis indica, and Cannabis ruderalis). The most commonly known phytocannabinoid is tetrahydrocannabinol (THC). However, there are more than sixty known phytocannabinoids including cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV) and cannabigerol monoethyl ether (CBGM).

Endogenous cannabinoids (also known as endocannabinoids) are compounds found in the nervous and immune systems of animals which activate cannabinoid receptors. Endogenous cannabinoids include compounds such as anandamide, docosatetraenylethanolamide and homo-γ-linoenylethanolamide, 2-arachidonyl glycerol, palmitoyl ethanolamide and oleamide.

Synthetic cannabinoids refer to cannabinoids that have been synthesized in a laboratory or cannabinoids which do not occur naturally. Synthetic cannabinoids include compounds such as JWH-018, CP-55940, dimethylheptylpyran, HU-210, HU-331, SR144528, WIN 55,212-2 , JWH-133 and levonantradol (nantrodolum).

As used herein, references to “consuming” a compound or “consumption” of a compound may refer to any known, or later developed, manner in which a compound may be administered or in which a compound may be consumed. For example, exemplary manners of consuming a compound may refer to smoking, orally ingesting, sublingual absorption, intravenous administration, etc.

As used herein, the term “patient” may refer to any human subject and/or animal subject.

As used herein, administering a compound in a “therapeutically effective amount” refers to administering a compound in any amount that will be effective to reduce or eliminate one or more of the side effects associated with consuming a cannabinoid. The dosage administered, as single or multiple doses, to an individual may vary depending upon a variety of different factors, including the method of administration, conditions and characteristics of the patient (e.g., sex, age, body weight, health, etc.), extent of symptoms, potency of compounds, concurrent treatments, frequency of treatment and the effect desired. Adjustment and manipulation of established dosage ranges are well within the ability of those skilled in the art. All of the compounds disclosed herein may be provided in the present compositions in a therapeutically effective amount.

As used herein, a “pharmaceutically acceptable” carrier encompasses any carrier, which does not interfere with effectiveness of the biological activity of the active ingredient and that is not toxic to the host to which it is administered. All of the compositions disclosed herein may include or be combined with a pharmaceutically acceptable carrier.

It should also be noted that although the compounds disclosed below may be described as being part of daytime formula or nighttime formula, the present principles encompass any and all combinations of the compounds listed below. In addition, it should also be recognized that all of the compositions described below may be supplemented with additional compounds for a variety of different reasons (e.g., to improve taste).

### Daytime Formula

As explained above, the consumption of marijuana or cannabinoids is associated with a number of different side effects. The daytime formula aims at eliminating or mitigating those side effects which have a negatively impact the daily operations of patient. For example, the compounds in the daytime formula may address the adverse impact that cannabinoids have on a patient's energy, motivation, productivity, memory and/or concentration. The daytime formula permits a patient to maintain his or her professional life and effectively carry out daily operations, despite the fact that the patient must consume cannabinoids for treating an illness or disease.

The composition of the daytime formula combines at least one cannabinoid (e.g., THC) with at least one of the following compounds: vitamin B complex, vitamin C, citicoline, guarana, L-Alpha Glycerylphosphorylcholine, ginseng, gingko biloba, taurine, caffeine, eleuthero, ginger, St. Johns wart, spirulina, kola nut, yerba mate, schisandra, bromelain, choline, maca root, 1-ornithine, lecithin, rhodiala, ornithine alpha ketoglutarate, acai, tyrosine, centella asiatica or rosemary.

Each one of the above compounds has been strategically selected to offset one or more of the adverse effects brought on by consuming THC, or other cannabinoids. For example, to offset the lethargy or lack of motivation associated with the consuming cannabinoids, the daytime formula may include caffeine, guarana, or eleuthero. To offset the negative effects that cannabinoids have on concentration, focus and brain function, the daytime formula may include ginseng, gingko biloba, taurine, L-Alpha Glycerylphosphorylcholine or citicoline. Vitamin B complex, vitamin C, eleuthero and St. John's wart may be included to offset the mood-related side effects and side effects related to anxiousness. To assist in the fast absorption of cannabinoids and other ingredients, the daytime formula may further include ginger or spirulina. Furthermore, certain compounds (such as ginseng, gingko, etc.) may increase the blood flow of a patient, thus enabling the active ingredients of the composition to be distributed throughout the patient's body relatively quickly. Since the compounds are being pushed through the patient's body faster than normal, the desired effects/benefits of compounds can also be felt faster than normal.

It should be recognized that the present principles may include the above compounds in any amount or dosage. However, the preferred amounts of each compound are provided below. The ranges for each compound provided below refer to the amount of a compound which is included in a single product (e.g., in a single pill, lollipop, etc).

In preferred embodiments, THC is selected as the cannabinoid for use in the daytime formula, and this compound is provided in the range of 1-1000 milligrams (mg), are more preferably in the range of 350-500 mgs. THC-based compounds may be administered to patients for a number of reasons and to treat a wide variety of symptoms or illnesses. For example, medical marijuana including THC is often prescribed to patients undergoing cancer treatments in order to increase their appetite and to eliminate nausea. In addition, it may also be useful for blocking certain deposits in the brain that cause Alzheimer's disease, to stop the neurological effects and muscle spasms that come from multiple-sclerosis, to slow down the tics in those suffering from Tourette's syndrome, to deal with obsessive neurological symptoms in people with obsessive compulsive disorder, and to lower intraocular pressure in patients with primary open-angle glaucoma. Even further, the muscle relaxing effects of THC-based products may also be useful in treating abdominal pain, cramps, etc.

Vitamin B complex may be included to enhance a patient's mood and outlook. Vitamin B complex is comprised of several different vitamins. In preferred embodiments, the various vitamins may be supplied in following doses: folic acid (200-800 micrograms); biotin (5-100 micrograms); inositol (5-100 mg); PABA or para-aminobenzoic acid (5-100 mg); thiamine (50-100 mg); riboflavin (5-100 mg); niacin and/or niacinamide (5-100 mg); pantothenic acid (5-100 mg); pyridoxine hydrochloride (5-100 mg) and cyanocobalamin (5-10 micrograms).

Vitamin C can be included to enhance a patient's mood. In addition, vitamin C can help improve the functioning of the immune system. Since research has shown that medical marijuana, as well as other products embodying cannabinoids, tends to aggravate existing injuries, vitamin C may serve to boost a patient's immune system, and thus mitigate such aggravating effects. In preferred embodiments, 25 milligrams (mg) to 90 mg of vitamin C is included.

Citicoline (also known as cytidine diphosphate-choline, CDP-Choline and cytidine 5′-diphosphocholine) is a psychostimulant/nootropic which may improve focus and mental energy, increase densities of dopamine receptors, and improve memory impairment. Research suggests that consumption of this compound promotes brain metabolism by enhancing the synthesis of acetyl-choline, restoring phospholipid content in the brain and affecting neuronal membrane excitability and osmolarity. In preferred embodiments, citicoline is provided within the range of 1-1500 mg.

L-alpha glycerylphosphorylcholine (also known as alpha GPC or choline alfoscerate) increases brain function and assists in learning. Thus, L-alpha glycerylphosphorylcholine may be included to combat the negative effects that a cannabinoid may have on focus, concentration or memory. In preferred embodiments, L-alpha glycerylphosphorylcholine is provided in the range of 300 mg-1,200 mg.

Ginseng, gingko biloba and taurine may assist in rebuilding and/or increasing a patient's concentration, focus and motivation. In preferred embodiments, 250 mg-500 mg of ginseng is included, 20-80 mg (60 mg) of ginkgo biloba is included and 20-1000 mg of taurine is included. It should be noted that the term “ginseng” as used herein encompasses at least the following compounds: Asian ginseng, panax ginseng, Korean ginseng, wild ginseng root (panax quinquefolium), rainey ginseng, cultivated Canadian ginseng, American ginseng, Siberian ginseng (eleutherococcus senticosus), white ginseng, pulse powder (shen mai san), codonopsis, wild ginseng, ralia, schefflera, tropical snowflake ginseng, Chinese ginseng, Korean red ginseng, red ginseng extract, ginseng royal jelly, pine brand ginseng, il hwa ginseng, ginseng CA meyer, Oriental ginseng, red panax ginseng, panax trifolium, panax notoginseng, panax japonicum, red dock ginseng, China white ginseng, panax notoginseng, Asiatic ginseng, ren shen, jiln ginseng, dwarf ginseng, himalayn ginseng, tien-chi and Brazilian ginseng.

In some embodiments, eleuthero is the preferred form of ginseng to be used. Eleuthero (also known as eleutherococcus senticosus, acanthopanax senticosus or Siberian ginseng) provides a number of different benefits. It helps to reduce fatigue, stress and depression, while enhancing neuron activity and improving memory. In addition, eleuthero is an immunogenic which serves to protect a patient from the radiation and chemicals used in a variety of different cancer treatments. Since many patients consume cannabinoids as part of a cancer treatment, compositions including eleuthero may be particularly advantageous for these patients. In preferred embodiments, eleuthero is provided in the range of 550 mg-900 mg.

Guarana and/or caffeine may be included in the daytime formula to provide energy and to combat tiredness and fatigue. It should be recognized that guarana inherently includes caffeine. Thus, in certain embodiments, wherein guarana is included, it may not be necessary to include additional caffeine. In preferred embodiments, guarana is provided in the range of 150 mg-1 gram (g), and caffeine is provided in the range of 50 mg-500 mg.

Ginger enables faster absorption of compounds in the stomach and helps to fight dyspepsia which is a common symptom of many cancer treatments. In preferred embodiments, ginger is provided in the range of 150 mg-1000 mg.

St. John's wort (also known as Hypericum perforatum) enhances an individual's mood and fights depression in a patient. In preferred embodiments, this compound may be included in the range of 100 mg-400 mg.

Yerba mate (also known as ilex paraguariensis) stimulates focus and clarity, and boosts an individual's energy and immune system. In preferred embodiments, yerba mate is included in the range of 400 mg-1500 mg.

Spirulina (also known as arthrospira platensis, macroalgae, and phytoplankton) strengthens a patient's immune system, enhances natural cleansing and detoxification and improves gastrointestinal and digestive health. In preferred embodiments, spirulina may be provided in the range of 1 microgram (mcg) to 3 grams.

Kola nut (also known as cola) enhances alertness and physical energy, elevates mood and increases tactile sensitivity. In preferred embodiments, this compound may be provided in the range of 1 mcg-3 g.

Schisandra (also known as magnolia vine) includes all plants in the schisandra genus including, but not limited to, schisandra chinensis. Plants in this genus have a protective effect on the liver, stimulate the immune system, increase stamina, decrease fatigue and provide relief for dry mouth symptoms. In preferred embodiments, these compounds may be provided in the range of 1 mcg-10 g.

RoseMary (rosmarinus officinalis) may enhance a patient's memory. In preferred embodiments, this compound may be provided in the range of 1 mcg-3 g.

Maca root (lepidium meyenii) may provide stamina and endurance, as well as promote mental clarity. In preferred embodiments, this compound may be provided in the range of 1 mcg-3 g.

Choline assists in brain development, prevents memory loss and protects against certain types of cancers. In preferred embodiments, this compound is provided in the range of 100 mg-1000 mg.

Lecithin is naturally occurring compound which is found in all cells. This compound improves memory function, plays an important role for overall nutrition, and has beneficial effects on liver function and cardiovascular health. This compound contains naturally occurring choline, inositol and essential fatty acids. Since vitamin B3 (aka Niacin) depletes choline and lecithin levels within body, this compound may be particularly advantageous in compounds which comprise vitamin B3. In preferred embodiments, this compound is provided in the range of 1 mcg-50 g.

L-ornithine may improve healing and promotes the production of the human growth hormone (HGH). In preferred embodiments, this compound is provided in the range of 1 mcg-5 g.

Bromelain, or any pineapple extract which includes bromelain, may also be included in the daytime formula. Bromelain increases digestive absorption, provides benefits for some intestinal disorders, and others believe may even serve as a pain reliever. In preferred embodiments, this compound may be provided in the range of 1 mcg-3 g.

Rhodiala (also known as golden root, roseroot, roseroot crenulin, Russian rhodiola, rhodiola rosea) has a number of beneficial effects. This compound has been used to treat depression and fatigue, and to help a patient's body respond more efficiently to stress caused by chemical, biological and physical variables. It also helps to increase the brain's betaendorphins and to reduce insomnia, irritability, hypertension, depression and headaches. Even further, it may be used to strengthen the immune system. In preferred embodiments, this compound may be provided in the range of 100 mg-500 mg.

Ornithine alpha ketoglutarate (also known as ornithine alpha-ketoglutarate, AKG, alpha-ketoglutarate, OKG, OAKG, Ornicetil, L-Ornithine Alpha-Ketoglutarate, L(+)-ornithine alpha-ketoglutarate) is a compound which enhances performance in catabolic conditions associated with physical activities (e.g., such as sports and weightlifting). It improves endurance, decreases muscle fatigue and speeds up recovery time. In addition, it increases production of human growth hormone (HGH) which in turn increases muscle mass. In preferred embodiments, this compound may be provided in the range of 0.5 g-15 g.

Acai (also known as assai, super berry, power berry, Viagra da amazonia, euterpe oleacea, acai berry) enhances digestive capabilities of the body, improves bowel movements, serves as an anti-cancer stimulant and helps regulate blood circulation. In preferred embodiments, this compound may be provided in the range of 200 mg-1500 mg.

Tyrosine (also known as 4-hydroxphenylalanine, acetyl-1-tyrosine, 1-tyrosine, n-acetyl 1-tyrosine, n-acetyl-tyrosine, tirosina, tyr, tyrosinum, 2-amino-3-(4-hydroxyphenyl) propionic acid) is a mood enhancer which can be used to treat depression. This compound also has antioxidant effects that prevents the formation of cancer. In preferred embodiments, this compound may be provided in the range of 100 mg-1500 mg.

Centella asiatica (Gotu Kola) is a stimulant which can be used to increase an patient's energy. In preferred embodiments, this compound may be provided in the range of 1 mg-1500 mg.

### Nighttime Formula

THC, and other cannabinoids, negatively affects a patient's sleep patterns by decreasing levels of slow wave sleep (SWS) and rapid eye movement (REM) sleep. In addition, cannabinoids may further hamper a patient's sleep by causing anxiety or paranoia in a patient. The nighttime formula allows a patient to enjoy the beneficial effects of cannabinoids, while eliminating or mitigating the negative effects that cannabinoids have on a patient's sleep.

The composition of the nighttime formula combines at least one cannabinoid (e.g., THC) with at least one of the following compounds: ginger, melatonin, galanthamine, huperzine A, vitamin B5, vitamin B6, valerian root, 5-Hydroxytryptophan, folic acid, 1-arginine, choline, spirulina, damiana, phenylalanine, bromelain, lecithin, ornithine alpha ketoglutarate, acai, tyrosine and rosemary.

It should be recognized that the present principles may include the above compounds in any amount or dosage. However, the preferred amounts of each compound are provided below. The ranges for each compound provided below refer to the amount of a compound which is included in a single product (e.g., in a single pill, lollipop, etc). As evidenced by the list above, several of the compounds used in the daytime formula may also be used in the nighttime formula. For these compounds that are common to both the daytime and nighttime formulas, the preferred dosages for the daytime formula also apply to the nighttime formula unless noted otherwise.

Melatonin (also known as N-acetyl-5-methoxytryptamine) increases REM sleep time and dream activity, and has been used to treat of certain forms of insomnia. In addition, melatonin improves the effectiveness of certain cancer medications used to fight tumors in the breast, lung, kidney, liver, pancreas, stomach, colon, prostate, and decreases some side effects associated with cancer treatments. In preferred embodiments, melatonin is provided in the range of 0.3 mg-20 mg.

Galantamine may be included to improve memory and to assist a patient in entering dream states. This compound improves the ability to think and remember or slows the loss of these inabilities, and has been used to treat adults with brain damage, Alzheimer's disease and dementia. In preferred embodiments, this compound is provided in the range of 2 mg-24 mg.

Vitamin B5 (also known as pantothenic acid) boosts immunity in patients and helps to fight stress. In preferred embodiments, this compound is provided in the range of 75 mg-1000 mg.

Vitamin B6 helps produce serotonin and melatonin which can help a patient sleep. In preferred embodiments, this compound is provided in the range of 10 mg-500 mg.

Valerian root has been used to treat insomnia and other disorders, and acts as a sedative for nervous tension, hysteria, excitability and stress. In preferred embodiments, this compound is provided in the range of 50 mg-2000 mg.

5-Hydroxytryptophan (also known as oxitriptan and 5-HTP) increases serotonin levels in the brain. It improves sleep quality and reduces pain, stiffness, anxiety and depression. 5-Hydroxytryptophan also increases leptin, which plays important role in appetite and other physiological processes. In preferred embodiments, this compound is provided in the range of 50 mg-1000 mg.

Folic acid (also known as folacin and vitamin B9) increases the appetite, stimulates the formation of digestive acids and can combat insomnia. In preferred embodiments, this compound is provided in the range of 45 mg-500 mg.

L-arginine (also known as L-Arginine Hydrochloride) may also be included in the nighttime formula. This compound may stimulate the body's production of growth hormone. In preferred embodiments, this compound is provided in the range of 1 mg-5 g.

Phenylalanine is used to treat chronic pain by stimulating nerve pathways in the brain that control pain. It enhances production of dopamine and norepinephrine. In preferred embodiments, this compound is provided in the range of 100 mg-1000 mg.

Damiana has a relaxing effect on the central nervous system. In preferred embodiments, this compound is provided in the range of mcg-5 g.

Huperzine A, or other huperzia serrata extract or derivative, may also be used in the nighttime formula. These compounds increase the production of acetylcholine which helps an individual to maintain sleep. In preferred embodiments, this compound is provided in the range of 1 mcg-1 g.

### Administration of Compositions

The compositions disclosed in this application may be administered in any known manner. Routes of administration include, but are not limited to, oral, intracranial ventricular, intrathecal, intravenous, parenteral, rectal, topical ophthalmic, subconjunctival, nasal, aural, sub-lingual (under the tongue) and transdermal, etc.

While the compounds listed above may be combined with cannabinoids in any dosage, in preferred embodiments, cannabinoids and compounds are provided in therapeutically effective amounts. Moreover, it should be appreciated that the present compositions may be administered with any suitable carriers, excipients, and other agents that are incorporated into formulations to provide improved transfer, delivery, tolerance, and the like.

The compositions described herein can be embodied in many forms, including but not limited to solid, semi-solid and liquid dosage forms such as tablets, pills, powders, liquid solutions or suspensions, injectable and infusible solutions, or a metered dose inhaler. In preferred embodiments, the present compositions are incorporated into edible foods or lollipop/sucker candies to permit sublingual absorption.

Having described preferred embodiments of the present principles (which are intended to be illustrative and not limiting), it is noted that modifications and variations can be made by persons skilled in the art in light of the above teachings. Therefore, it is to be understood that while certain changes may be made to the particular embodiments disclosed herein, these changes are within the scope of the present invention as outlined by the appended claims. Having thus described aspects of the present invention, with the details and particularity required by the patent laws, what is claimed and desired protected by Letters Patent is set forth in the appended claims.

What is claimed is:

1. A composition, comprising:
at least one cannabinoid; and
at least one of the following compounds: vitamin B complex, vitamin C, citicoline, guarana, 1-alpha glycerylphosphorylcholine, ginseng, gingko biloba, taurine, caffeine, eleuthero, ginger, and St. Johns wart.

2. The composition of claim 1, further comprising at least one of spirulina, kola nut, yerba mate, schisandra, bromelain, rhodiala and rosemary.

3. The composition of claim 1, wherein:
vitamin C is provided within a range of 25-90 milligrams (mg),
citicoline is provided within a range of 150 mg to 1 gram (g),
1-alpha glycerylphosphorylcholine is provided within a range of 300-1,200 mg,
ginseng is provided within a range of 250 mg-500 mg,
ginkgo biloba is provided within a range of 20 mg-80 mg,
taurine is provided within a range of 20 mg-1000 mg,
guarana is provided within a range of 150 mg-1 g,
caffeine is provided within a range of 50 mg-500 mg,
eleurthero is provided within a range of 550 mg-900 mg,
ginger is provided within a range of 150 mg-1000 mg, and
St. John's wort is provided within a range of 100 mg-400 mg.

4. The composition of claim 2, wherein:
spirulina is provided within a range of 1 microgram (mcg) up to 3 grams (g),
kola nut is provided within a range of 1 mcg-3 g,
schisandra is provided within a range of 1 mcg-10 g,
rosemary is provided within a range of 1 mcg-3 g,
rhodiala is provided within a range of 100 milligrams (mg) to 5000 milligrams,
maca root is provided within a range of 1 mcg-3 g, and
bromelain is provided within a range of 1 mcg-3 g.

5. The composition of claim 1, further comprising at least one of choline, maca root, 1-ornithine, ornithine alpha ketoglutarate, acia, tyrosine, centella asiatica and lecithin.

6. The composition of claim 5, wherein:
choline is provided within a range of 100 milligrams (mg)-1000mg,
maca root is provided within a range of 1 microgram (mcg) to 3 grams (g),
ornithine alpha ketoglutarate is provided within a range of 0.5 g-15 g,
1-ornithine is provided within a range of 1 mcg-5 g,
acia is provided within a range of 200-1500 mg,
tyrosine is provided within a range of 100 mg-1500 mg,
centella asiatica is provided within a range of 1 mg-1500 mg, and
lecithin is provided within a range of 1 mcg-50 g.

7. The composition of claim 1, wherein said composition is embodied in an edible food product.

8. The composition of claim 7, wherein said edible food product comprises a lollipop or sucker permitting sublingual absorption.

9. The composition of claim 1, wherein said composition is embodied in either a pill or solution.

10. A composition, comprising:
at least one cannabinoid; and
at least one of the following compounds: ginger, melatonin, galanthamine, huperzine A, vitamin B5, vitamin B6, choline, valerian root, 5-Hydroxytryptophan and folic acid.

11. The composition of claim 10, further comprising at least one of 1-arginine, spirulina, damiana, bromelain, lecithin, phenylalanine, ornithine alpha ketoglutarate and rosemary.

12. The composition of claim 10, wherein:
ginger is provided within a range of 150 milligrams (mg)-1000 mg,
melatonin is provided within a range of 0.3 mg-20 mg,
galanthamine is provided within a range of 2 mg-24 mg,
huperzine A is provided within a range of 1 microgram (mcg)-1 gram (g),
choline is provided within a range of 100 mg-1000 mg,
vitamin B5 is provided within a range of 75 mg-1000 mg,
vitamin B6 is provided within a range of 10 mg-500 mg,
valerian root is provided within a range of 50 mg-2000 mg,
5-Hydroxytryptophan is provided within a range of 50 mg-2000 mg, and
folic acid is provided within a range of 45 mg-500 mg.

13. The composition of claim 11, wherein:
1-arginine is provided within a range of 100 milligrams (mg)-2000 mg,
spirulina is provided within a range of 1 microgram (mcg) up to 3 grams (g),
damiana is provided within a range of 1 mcg-5 g,
lecithin is provided within a range of 1 mcg-50 g,
bromelain is provided within a range of 1 mcg-3 g,
ornithine alpha ketoglutarate is provided within a range of 0.5 g-15 g,
rosemary is provided within a range of 1 mcg-3 g, and
phenylalanine is provided within a range of 100 mg-1000 mg.

14. The composition of claim 10, wherein said composition is embodied in an edible food product.

15. The composition of claim 14, wherein said edible food product comprises a lollipop or sucker candy permitting sublingual absorption.

16. The composition of claim 10, wherein said composition is embodied in either a pill or solution.

17. A method for administering a cannabinoid composition, comprising:
administering a therapeutically effective amount of a cannabinoid composition to a patient, wherein the cannabinoid composition comprises:
at least one cannabinoid; and
at least one of the following compounds: vitamin B complex, vitamin C, citicoline, guarana, 1-alpha glycerylphosphorylcholine, ginseng, gingko biloba, taurine, caffeine, eleuthero, ginger, St. Johns wart, melatonin, galanthamine, huperzine A, vitamin B5, vitamin B6, choline, valerian root, 5-Hydroxytryptophan and folic acid.

18. The method of claim 17, wherein the cannabinoid composition further comprises at least one of spirulina, kola nut, yerba mate, schisandra, bromelain and rosemary.

19. The method of claim 17, wherein the cannabinoid composition further comprises at least one of choline, maca root, 1-ornithine, and lecithin.
